(19) United States
(12) Patent Application Publication
(10) Pub. No.: US 2009/0130151 A1
(43) Pub. Date: May 21, 2009

(54) DISPERSION OF NATURAL OILS AND/OR SYNTHETIC ESSENTIAL OILS IN WATER

(75) Inventor: Richard Mark Pashley, Cook (AU)

Correspondence Address: DARBY & DARBY P.C., P.O. BOX 770, Church Street Station, New York, NY, 10008-0770, US

(73) Assignee: THE AUSTRALIAN NATIONAL UNIVERSITY, Canberra (AU)

(21) Appl. No.: 12/088,163

(22) PCT Filed: Sep. 28, 2006
(86) PCT No.: PCT/AU2006/001425

§ 371 Date: Aug. 26, 2008

(30) Foreign Application Priority Data
Sep. 28, 2005 (AU) 2005905337

(51) Int. Cl. A61K 8/06; C11D 17/00; B01F 13/00; B01F 13/06

(52) U.S. Cl. 424/401; 510/417; 366/342; 366/139

(57) ABSTRACT

The invention relates to process for the production of an emulsion, comprising combining an aqueous liquid with a non-aqueous liquid mixture to form a combination, removing dissolved gases from both the aqueous liquid and the non-aqueous liquid mixture and agitating the combination sufficient to form the emulsion. The non-aqueous liquid mixture is immiscible with the aqueous liquid, and combination is formed in the absence of an emulsion stabilizing agent.

### TECHNICAL FIELD

The present invention relates to a method for dispersing natural oils and/or synthetic essential oils in water and to dispersions of natural and/or synthetic essential oils in water.

### BACKGROUND OF THE INVENTION

Recent studies have demonstrated that pure hydrocarbon oils can be dispersed in water as fine droplets without the use of additives. The high interfacial tension between hydrocarbons and water is expected to cause cavitation between oil droplets during separation. This cavitation is aided by dissolved atmospheric gases present in both the oil and water. Their removal allows oil droplets to be readily dispersed in water.

A disadvantage with this method is that it relies on systems in which a high interfacial tension exists between the oil and the water. It would be an advantage to be able to readily form emulsions of natural oils and/or synthetic essential oils in water using a similar process. However natural oils and synthetic essential oils commonly have a quite low interfacial tension with water due to polar components in the oils. Consequently the above mechanism is not expected to apply.

### OBJECT OF THE INVENTION

It is the object of the present invention to substantially overcome or at least ameliorate the disadvantage.

### SUMMARY OF THE INVENTION

In a first aspect of the invention there is provided a process for the production of an emulsion, comprising:

combining an aqueous liquid with a non-aqueous liquid mixture that is normally immiscible with the aqueous liquid, to form a combination;

removing dissolved gases from both the aqueous liquid and the non-aqueous liquid mixture; and

agitating the combination sufficient to form the emulsion.

The non-aqueous liquid mixture may comprise at least two components having different interfacial tensions with water. It may comprise a mixture of said at least two components. The non-aqueous liquid mixture may have a theoretical water contact angle in air of less than about 90°. In this context the theoretical water contact angle is the contact angle of the water with an equivalent solid having the same wetting properties as the non-aqueous liquid mixture. The non-aqueous liquid mixture may be more polar than air. The non-aqueous liquid may have a lower interfacial tension with water than with air. The non-aqueous liquid mixture may be a non-polar liquid. It may be a hydrophobic liquid. It may be a low polarity liquid. The non-aqueous liquid mixture may be a natural oil. It may comprise or consist of a naturally occurring fragrant oil or a mixture of at least two naturally occurring fragrant oils (e.g. a mixture of 2-20, 2-15, 2-10, 2-5, 2-4, 2-3 or 2 naturally occurring fragrant oils). The non-aqueous liquid mixture may be a natural essential oil. The non-aqueous liquid mixture may be a synthetic essential oil. It may comprise or consist of an essential oil or a mixture of 2, 3 4, 5, 6, 7, 8, 9, 10, 11, 12, 13, 14, 15, 16, 17. 18, 19 or 20 or more essential oils which may be natural essential oils or synthetic essential oils. The mixture may be a mixture of one or more natural essential oils in combination with one or more synthetic essential oils. The mixture may be a mixture of two or more natural essential oils. The mixture may be a mixture of two or more synthetic essential oils. A natural essential oil is a water-immiscible liquid derived from plant material. An essential oil is alternatively referred to as a volatile oil or an ethereal oil. The non-aqueous liquid mixture may not be soybean oil. The non-aqueous liquid mixture may be for example eucalyptus oil, lavender oil, tea tree oil, bergamot oil, frankincense oil, patchouli oil, cedarwood oil, lime oil, peppermint oil, chamomile oil, grapefruit oil, mandarin oil, rose oil, ylang ylang oil, clary sage oil, jasmine oil, marjoram oil, cypress oil, juniper berry oil, neroli oil, rosewood oil, palmarosa oil, sandalwood oil, pine oil, mint oil, cinnamon oil, cajeput oil, fennel oil, geranium oil, girofle oil, lemon oil, spearmint oil, myrtle oil, oregano oil, ruse oil, rosemary oil, sarriette oil, thyme oil, hypericum oil, star anise seed oil, oil derived from cherry, orange, pineapple or orange, briar oil, violet oil, or garlic oil, or may comprise a mixture of any two, three, four, five, six, seven eight, nine, ten eleven, twelve, thirteen fourteen, fifteen, sixteen, twenty or more thereof. The essential oil may be an aromatic oil (e.g. lavendar oil, jasmine oil or camomile oil or any mixture thereof). The essential oil may include a substance which reduces the evaporation rate of volatile components in the oil (e.g. vanillin, or coumarin) The non-aqueous liquid mixture may comprise at least one volatile component. The volatile component may comprise or consist of one or more of an ester an aldehyde an alcohol, a ketone and a terpene, and may comprise a mixture of any two or more of these. The volatile component may be of plant origin. The non-aqueous liquid mixture may have low viscosity. It may have a viscosity of less than about 70 cP at 15° C., or less than about 65, 60, 55, 50, 45, 40, 35, 30, 25, 20, 15, 10 or 5 cP at 15° C., or between about 1 and about 70 cP, or 5 and 70, 10 and 70, 20 and 70, 5 and 50, 10 and 50, 10 and 40, 30 and 70, 40 and 70, 20 and 50, 20 and 30 or 30 and 50 cP at 15° C., e.g. about 1, 2, 3, 4, 5, 10, 15, 20, 25, 30, 35, 40, 45, 50, 55, 60, 65 or 70 cP at 15° C.

The non-aqueous liquid mixture may comprise or consist of non-polymeric components. It may comprise no polymeric or oligomeric components. The non-aqueous liquid may not be a surfactant. It may possess negligible or no surfactant properties.

The combination may be formed in the absence of an emulsion stabilizing agent. The emulsion produced by the process may be stable for a period of from one hour to several weeks.

The step of removing dissolved gases may comprise removing at least 90 percent of dissolved gas present in the combination, the aqueous liquid or the non-aqueous liquid mixture. It may comprise exposing the combination to a vacuum. It may comprise first freezing the combination and then exposing the frozen combination to a vacuum (i.e. a freeze-thaw cycle). It may comprise a plurality of successive freeze-thaw cycles. The vacuum may have an absolute pressure of less than about twenty Torr.

The non-aqueous liquid mixture may comprise or consist of a triglyceride and/or a natural oil.

The invention also provides an emulsion when made by the process of the first aspect. The emulsion may have a mean droplet size of less than about 10 microns.

In a second aspect of the invention there is provided an emulsion comprising a non-aqueous liquid mixture dispersed in an aqueous liquid, said non-aqueous liquid mixture comprising at least two components having different interfacial tensions with water and said emulsion having no emulsion stabilising agent, wherein the emulsion is stable for at least one hour. The emulsion may have a mean droplet size of less than about 10 microns.

In a third aspect of the invention there is provided a preparation for use in maintaining or improving personal hygiene, said preparation comprising an emulsion made by the process of the first aspect of the invention, or comprising an emulsion according to the second aspect of the invention.

In a fourth aspect of the invention there is provided an apparatus for preparing an emulsion of a non-aqueous liquid mixture in an aqueous liquid, said apparatus comprising:

a mixing chamber for combining the aqueous liquid with the non-aqueous liquid mixture to form a combination;

at least one degasser for removing dissolved gas from both the aqueous liquid and the non-aqueous liquid mixture; and

an agitator for agitating the combination to form the emulsion.

### BRIEF DESCRIPTION OF THE DRAWINGS

A preferred embodiment of the present invention will now be described, by way of an example only, with reference to the accompanying drawings wherein:

FIG. 1 shows a graph of turbidity measurements for de-gassed and gassed (blank) tea tree oil in water as a function of time after shaking (refractive index of oil: 1.4769);

FIG. 2 shows particle size data on degassed dispersions of 0.2 ml of Tea Tree oil in 25 ml of water (Diam. (nm): 1800, Z-Average size (nm): 1600, PDI (polydispersity index): 0.123);

FIG. 3 shows particle size data on tea tree oil dispersions, for gassed (blank) dispersions of 0.2 ml of Tea Tree oil in 25 ml of water, illustrating that no sensible values could be obtained from the gassed (blank) samples, as the droplets were large and polydispersed (Diam. (nm): 0.00, Z-Average size (nm): 1.13×10⁴, PDI: 1.000);

FIG. 4 shows turbidity measurements on eucalyptus oil dispersed in water under gassed (blank) and de-gassed conditions (refractive index of eucalyptus oil: 1.4603);

FIG. 5 shows particle size measurements on eucalyptus oil dispersed in water under de-gassed conditions (Diam. (nm): 2220, Z-Average size (nm): 3590, PDI: 0.330);

FIG. 6 shows turbidity measurements of lavender oil under de-gassed and gassed conditions (refractive index: 1.4690);

FIG. 7 shows particle size measurements on de-gassed samples of lavender oil and water (Diam. (nm): 3180, Z-Average size (nm): 2880, PDI: 0.194); and

FIG. 8 shows photographs of lavender oil and water taken minutes after shaking, in which the de-gassed combination is on the left.

### DETAILED DESCRIPTION OF THE PREFERRED EMBODIMENTS

The present invention relates to the effect of de-gassing on the dispersion of bicomponent or multicomponent water-immiscible liquids such as natural oils. These natural, mixed oils include for example eucalyptus, lavender and tea tree oil. Although these oils are mixtures and in some cases not as hydrophobic as those used in earlier studies, the effect of de-gassing substantially enhances their dispersion, producing micron-sized droplets without the need for additives. Dispersions of these oils in pure water have a wide range of uses where purity is an advantage, for example, in skin cleaning products and oral sprays.

Thus the present invention discloses a process for the production of an emulsion, comprising combining an aqueous liquid with a non-aqueous liquid mixture to form a combination, removing dissolved gases from both the aqueous liquid and the non-aqueous liquid mixture, and agitating the combination sufficient to form the emulsion. The non-aqueous liquid mixture is normally immiscible with the aqueous liquid, and comprises at least two components having different interfacial tensions with water.

It will be understood that most liquids are to some degree miscible with most other liquids. In the present context two liquids may be taken to be immiscible if, when equal volumes of the two liquids are agitated together vigorously and then allowed to completely equilibrate without agitation, neither of the two liquids will be present in the other liquid at a concentration of greater than about 10%, or greater than about 1, 1, 0.5, 0.2 or 0.1%.

The emulsion may be an oil-in-water (O/W) emulsion. In an O/W emulsion, the non-aqueous liquid mixture is the dispersed phase of the emulsion and the aqueous liquid is the continuous phase of the emulsion.

The ratio of the aqueous liquid to the non-aqueous liquid mixture may be between about 1 and about 1000 (i.e. between about 1:1 and about 1000: 1), or between about 5 and 1000, 10 and 1000, 20 and 1000, 30 and 1000, 40 and 1000, 50 and 1000, 60 and 1000, 70 and 1000, 80 and 1000, 90 and 1000, 200 and 1000, 300 and 1000, 400 and 1000, 500 and 1000, 1 and 500, 1 and 200, 1 and 100, 1 and 50, 1 and 20, 1 and 10, 10 and 500, 20 and 200, 50 and 200, 50 and 150, 80 and 120 or 90 and 110, e.g. about 1, 1.5, 2, 2.5, 3, 3.5, 4, 4.5, 5, 6, 7, 8, 9, 10, 15, 20, 25, 30, 35, 40, 450, 50, 60, 70, 80, 90, 95, 100, 105, 110, 115, 120, 130, 140, 150, 160, 170, 180, 190, 200, 250, 300, 350, 400, 450, 500, 600, 700, 800, 900 or 1000, or may be less than 1 or greater than 1000. The proportion of the non-aqueous liquid mixture in the emulsion may be between about 0.1 and about 50%, or between about 0.5 and 50, 1 and 50, 10 and 50, 20 and 50, 0.1 and 20, 0.1 and 10, 0.1 and 5, 0.1 and 2, 0.1 and 1, 0.1 and 0.5, 0.5 and 20, 0.5 and 10, 0.5 and 5, 0.5 and 2 or 0.5 and 1.5, and may be about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 25, 30, 35, 40, 45 or 50% (w/w or w/v).

Once the emulsion has been formed according to the present invention, water may be removed, e.g. by evaporation, to increase the proportion of the non-aqueous liquid mixture in the emulsion. This may be conducted without substantial change in the droplet size (e.g with an increase in mean droplet size, or of the maximum droplet size or of the number average, weight average or Z-average droplet size) of less than about 20%, or less than about 10, 5, 2 or 1%. The water may be removed to achieve a final water content of the emulsion of between about 10 and about 70% by volume, or between about 10 and 50, and 30, 30 and 70, 50 and 70, 30 and 60 or 40 and 60%, e.g. about 10, 15, 20, 25, 30, 35, 40, 45, 50, 55, 60, 65 or 70%.

The time and vigour of the agitation should be sufficient to form the emulsion. These may depend on the nature of the aqueous liquid and the non-aqueous liquid mixture, on the interfacial tension between them, on the ratio of the volumes of each and on the total volume of the combination. The time may depend on the vigour of the agitation. It may be for example at least about 1s, or at least about 2, 3, 4, 5, 10, 15, 20, 25, 30, 35, 40, 45, 50 or 55 seconds, at least about 1, 1.5, 2, 2.5, 3, 3.5, 4, 4.5, 5, 6, 7, 8, 9, 10, 15, 20, 25, 30, 40 or 50 minutes, or at least about 1, 1.5, 2, 2.5, 3, 3.5, 4, 4.5 or 5 hours, and may be about 1, 2, 3, 4, 5, 10, 15, 20, 25, 30, 35, 40, 45, 50 or 55 seconds, about 1, 1.5, 2, 2.5, 3, 3.5, 4, 4.5, 5, 6, 7, 8, 9, 10, 15, 20, 25, 30, 40 or 50 minutes, or about 1, 1.5, 2, m 2.5, 3, 3.5, 4, 4.5 or 5 hours. The agitation may be moderate, vigorous, very vigorous or violent. It may comprise shaking, swirling, stirring, sonicating, ultrasonicating or some other suitable agitation, or may comprise a combination of two or more of these.

The aqueous liquid may be water or a solution of one or more solutes in water. The solutes may be salts or other suitable solutes. The concentration and nature of the solutes should be such that the aqueous liquid is immiscible with the non-aqueous liquid mixture at the temperature at which the process is conducted. The temperature may be conducted at any temperature at which both the aqueous liquid and non-aqueous liquid mixture are liquid and at which they are immiscible. Suitable temperatures, depending on the nature of the two liquids, may be between about 0 and about 100° C., or between about 1 and 80, 0 and 60, 0 and 40, 0 and 20, 0 and 10, 10 and 100, 20 and 100, 40 and 100, 60 and 100, and 60, 10 and 40 or 15 and 30° C., e.g. about 0, 5, 10, 15, 20, 25, 30, 35, 40, 45, 50, 55, 60, 65, 70, 75, 80, 85, 90, 95 or 100° C. The aqueous liquid may not comprise an emulsion stabilising agent. Particular emulsion stabilising agents that may be absent from the aqueous liquid include surfactants (ionic, non-ionic or zwitterionic), particulate emulsion stabilisers, polymeric emulsion stabilisers etc. These should also be absent from the non-aqueous liquid mixture. Thus the combination may be formed in the absence of an emulsion stabilizing agent.

When there is a mixture of oils they may be in any ratio which is suitable for the required purpose, such as a ratio in the range of 1:99 v/v to 99:1 v/v, when there are two oils in combination with one another when any ratio within that range will suit the desired end use, for example.

The non-aqueous liquid mixture may comprise at least two compounds with different interfacial tensions with water. The difference in interfacial tensions may be at least about 2, 4, 6, 8 or 10 mN/m, and may be for example about 2, 3, 4, 5, 6, 7, 8, 9, 10, 11, 12, 13, 14, 15, 16, 17, 18, 19 or 20 mN/m. The non-aqueous liquid mixture may have a low interfacial tension with water. It may comprise a component with a low interfacial tension with water in sufficient quantity that the non-aqueous liquid mixture has a low interfacial tension with water. The interfacial tension between the non-aqueous liquid mixture and water may be less than about 40 mN/m, or less than about 35, 30, 25 or 20 mN/m, or may be about 10, 11, 12, 13, 14, 15, 16, 17, 18, 19, 20, 22, 24, 26, 28, 30, 35 or 40 mN/m. The theoretical water contact angle of the non-aqueous liquid mixture in air may be about 900, or less than 89, 88, 87, 86 or 850, and may be about 75, 76, 77, 78, 79, 80, 81, 82, 83, 84, 85, 86, 87, 88 or 890. The non-aqueous liquid mixture may be more polar than air. The non-aqueous liquid may have a lower interfacial tension with water than with air.

The emulsion produced by the process may be stable for a period of from one hour to several weeks. It may be stable for at least about 1, 2, 3, 4, 5, 6, 9, 12, 15 or 18 hours, or at least about 1, 1.5, 2, 2.5, 3.5, 4, 4.5, 5, 6, 7, 8, 9, 10, 11, 12 or 13 days, or at least about 2, 3, 4, 5, 6, 7, 8, 9 or 10 weeks. In this context, “stable” refers to a situation in which no gross separation of the hydrophobic liquid occurs when the emulsion is allowed to stand undisturbed for the stated period, or when the emulsion is gently agitated for the stated period. The emulsion may be such that after the period described above, the turbidity ratio is at least about 2, or at least about 2.5, 3, 3.5, 4, 4.5, 5, 6, 7, 8, 9 or 10. In this context the turbidity ratio is obtained by determining the turbidity of the emulsion to obtain a degassed turbidity and of an emulsion obtained using the same components and procedure but without degassing the aqueous liquid, the non-aqueous liquid mixture or the combination to obtain an undegassed turbidity, and then dividing the gassed turbidity by the undegassed turbidity. If two phases exist, the turbidity of the phase having the higher water content should be measured. The turbidity may be measured at 900 of scatter.

The step of removing dissolved gases may comprise removing at least about 90 percent of dissolved gas present in the combination, the aqueous liquid or the non-aqueous liquid mixture. It may comprise removing at least about 95, 96, 97, 98, 99, 99.5, 99.9, 99.95, 99.99, 99.995 or 99.999% of the dissolved gas present. It may comprise exposing the combination to a vacuum. It may comprise first freezing the combination and then exposing the frozen combination to a vacuum (i.e. a freeze-thaw cycle). It may comprise a plurality of successive freeze-thaw cycles (e.g. 2, 3, 4, 5, 6, 7, 8, 9 or 10). The vacuum may have an absolute pressure of less than about 20 Torr, or less than about 10, 5, 2, 1, 0.5, 0.2, 0.1, 0.05, 0.02, 0.01, 0.005, 0.002 or 0.001 Torr, or an absolute pressure of about 20, 10, 5, 2, 1, 0.5, 0.2, 0.1, 0.05, 0.02, 0.01, 0.005, 0.002 or 0.001 Torr. The freeze-thaw cycle is a well known means for degassing, and involves freezing the liquid, then applying a vacuum, then sealing the container with the liquid therein, then allowing the frozen liquid to thaw and thereby degas, then refreezing the liquid (preferably with minimal agitation) and repeating the cycle the desired number of times. Other processes for degassing that may be used include membrane degassing and sparging with a gas of low solubility in the liquid.

The process of the present invention may be applied to fragrant oils, aromatic oils, natural essential oils or synthetic essential oils or to oils having fragrant components or any mixture thereof. These oils have volatile materials which may potentially be lost during the stage of degassing, particularly degassing using a vacuum. It is therefore surprising that the present invention may be applied to such oils whereby the resulting emulsion has detectable fragrant and/or aromatic properties of the oil.

The step of degassing may be conducted before, during or after the step of forming the combination. Thus the aqueous liquid and the non-aqueous liquid mixture may be combined to form a combination and the combination may be degassed. Alternatively the aqueous liquid and the non-aqueous liquid mixture may be degassed and then combined (preferably under conditions that prevent or inhibit uptake of gas by the degassed liquids) to form a degassed combination, or one of the liquids may be degassed, then combined with the other liquid to form a combination, and the combination may then be degassed. The degassing should occur before or during the step of agitating to form the emulsion.

The non-aqueous liquid mixture may comprise a triglyceride and/or a natural oil.

Emulsions made by the process of the invention comprise a non-aqueous liquid mixture dispersed in an aqueous liquid, said non-aqueous liquid mixture comprising at least two components having different interfacial tensions with water and said emulsion having no emulsion stabilising agent, wherein the emulsion is stable for at least one hour. The mean droplet size may be less than about 10 microns, or less than about 9, 8, 7, 6, 5, 4, 3, 2 or 1 microns. The mean zeta potential of the droplets of the non-aqueous liquid in the emulsions of the present invention may be greater than about −50 mV, and may be for example about −49, −48, −47, −46, −45, −44, −43, −42, −41, −40, −39, −38, −36, −36 or −35 mV.

An emulsion according to the present invention may be a component in a preparation for use in maintaining or improving personal hygiene. The preparation may be for example a skin cleaning product (a soap, a liquid soap, a body lotion, a skin cleanser etc.), an oral spray, a shaving cream, a moisturising cream or lotion, a cosmetic preparation or some other type of preparation.

The invention also provides an apparatus for preparing an emulsion. The apparatus comprises:

a mixing chamber for combining the aqueous liquid with the non-aqueous liquid mixture to form a combination;

at least one degasser for removing dissolved gas from both the aqueous liquid and the non-aqueous liquid mixture; and

an agitator for agitating the combination to form the emulsion.

The agitator may be located in the mixing chamber, or may be located in a separate agitation chamber, said agitation chamber communicating with the mixing chamber in a manner that prevents or inhibits uptake of gas by a liquid or combination passing from the mixing chamber to the agitation chamber. A degasser may be located between the mixing chamber and the agitation chamber for degassing a liquid or combination passing from the mixing chamber to the agitation chamber. Alternatively or additionally a degasser may be located in the mixing chamber, or may be fitted to the mixing chamber. Alternatively or additionally there may be one or more degassers disposed to degas a liquid which enters the mixing chamber. The mixing chamber may comprise one or more inlet ports for admitting the liquids thereto. The agitator may comprise a stirrer, a blender, a sonicator, an ultrasonicator, a shaker, a disperser, a shear blade or some other suitable agitator. The degasser may for example comprise a membrane degassing unit, or may comprise suitable vacuum pump, valves and chiller for conducting freeze-thaw degassing.

Recent work has indicated that fine, micron sized droplets of oil may under certain circumstances be stable when dispersed in water as fine, micron-sized droplets. It is theorised that oil droplets become charged naturally in water due to the adsorption of hydroxyl ions. These charged droplets may then remain stable because of the weak van der Waals force between droplets. It has been suggested that the dispersion of fine hydrophobic droplets in water is inhibited by a cavitation process which is expected from an analysis of the thermodynamic processes involved in separating hydrophobic surfaces in water. The interfacial tension between hydrocarbons and water is higher then between hydrocarbons and air or vapour. This is the fundamental reason why separating these surfaces in water will cause cavitation. Further, cavitation creates several forces between the surfaces which will hold the separating droplets together and prevent their dispersion. These include the Laplace pressure and enhanced van der Waals forces, as well as the force opposing the extension of the bridging vapour cavity, as the droplets try to separate. However, the cavitation of water under ambient conditions requires a large input of energy. In practice it seems that dissolved gases in water act as nucleation sites for this cavitation process and hence removal of these gases inhibits cavitation and so enhances dispersion.

It is worthy of note that about 20 ml of atmospheric gases are dissolved in a litre of water under standard conditions of temperature and pressure. These gases can be removed almost completely, for example by freezing and thawing under a vacuum. Dispersions of hydrocarbon or fluorocarbon oils can be readily formed, on de-gassing, without the need for stabilizing surfactants and polymers. Earlier studies on the effects of de-gassing on dispersion were carried out on pure liquid hydrocarbons and fluorocarbon compounds. The key requirement appears to be that the oil should be both water insoluble and hydrophobic, for example, giving a high interfacial tension with water. Thus the high interfacial tension between hydrocarbons and water is expected to cause cavitation between oil droplets during separation. This is thought to be because the surface tension of the oil, in air or vapour, is lower than the interfacial tension between hydrocarbons and water. This cavitation may be aided by dissolved atmospheric gases present in both the oil and water. Their removal allows oil droplets to be readily dispersed in water.

The present invention extends this invention to other types of water immiscible oils, for example natural oils such as eucalyptus, lavender and tea tree oil. The significant effect of de-gassing on the dispersion of each of these natural oils in water is remarkable and especially interesting because of their interfacial tensions with both air and water. Cavitation is expected for pure hydrocarbon or fluorocarbon oil droplets, e.g. dodecane, separated in water, because of the low surface tension of dodecane (25.4 mN/m) and its high interfacial tension (52.9 mN/m) with water. That is, cavitation creates the lower energy oil/air interfaces, rather than the higher energy oil/water interfaces. However, it would not be expected that de-gassing less hydrophobic oils would enhance the dispersion of these oils because their surface tensions are higher than the corresponding interfacial tension with water. Consequently no driving force would appear to be present to form an oil/water interface in preference to an air/water interface, since the former interface would appear to be of higher energy.

Another way of considering this is through the concept of a theoretical water contact angle on an equivalent solid surface with the same wetting properties as the oil in air (i.e. using a droplet of water on such a solid surface). For example, using the Young equation for water droplets on dodecane, the theoretical contact angle is 112°. This high angle, >90°, indicates that air ‘wets’ the oil surface better than water. The oil is very hydrophobic. If the same analysis is applied to three less non-polar oils, tea tree oil, eucalyptus and lavender oil, theoretical water contact angles of 82°, 79° and 80° respectively may be calculated. Hence, although these oils are hydrophobic, they are not more hydrophobic than air and so de-gassing may be expected to have no effect. This is because the air/oil interface has a higher energy than the water/oil interface.

Vegetable oils have been found to be more readily dispersed in de-gassed water, even though the interfacial tension with water was low. The inventors hypothesis that this may be explained by the high molecular weight of soybean oil and the slight amphiphilic nature of the triple ester group. Thus, when measuring surface and interfacial tensions, large molecules can orientate to produce a lower tension. However, during vigorous shaking, the rapid dispersion process does not allow the high molecular weight molecules to orientate at the rapidly formed new surfaces of the droplets and so the system behaves much more like a pure liquid hydrocarbon. Consequently, although the oils used in the present invention may have a relatively low equilibrium interfacial tension, a transient interfacial tension may be considerably higher, and may be greater than the transient surface tension of the oil, thereby enabling degassing to facilitate emulsification.

This effect may occur for high molecular weight components of some natural oils, for example camphor. It is thought that a second effect may be caused by the presence of slightly polar component molecules, such as cineole, in a mostly non-polar liquid of, for example, α-pinene and terpinene. Polar components may diffuse to the surface or interface to lower the tension during static, equilibrium measurements. However, during rapid and vigorous shaking, this diffusion to the new surfaces formed will not be possible. Hence, again these multi-component oils would be expected to behave closer to pure hydrocarbons under these conditions, as described herein. This second effect is also related to the presence of a transient, relatively high interfacial tension between the oil and the aqueous liquid. This suggests that interfacial tension values are useful in predicting the effects of cavitation on dispersion, but only for pure, non-polar liquids. Thus in the present invention, many of the oils have relatively low molecular weight but are mixtures. The components may have a molecular weight less than about 1200, or less than about 1100, 1000, 900, 800, 700, 600, 500, 400, 300 or 200. The non-aqueous liquid mixture may have less than about 10% of material with molecular weight greater than about 1200 (or greater than about 1100, 1000, 900, 800, 700, 600, 500, 400, 300 or 200), or less than about 5%, 2% or 1% of such material. The non-aqueous liquid mixture may have less than about 10, 5 or 2% of material with molecular weight greater than about 200.

Some of the components of the mixtures are moderately polar. This has the effect of reducing the interfacial tension with water by selective adsorption of the polar components. However, during vigorous shaking there is insufficient time for this adsorption process and so these natural oil mixtures may behave more like hydrophobic, pure oils.

In both effects described above, it is important that the transient high interfacial tension is sufficiently long-lived that agitation to form an emulsion can occur before relaxation to the equilibrium low interfacial tension. Thus it is preferred that, following agitation of the aqueous liquid with the non-aqueous liquid mixture, the interfacial tension between the non-aqueous liquid mixture and the aqueous liquid remain greater than the surface tension of the non-aqueous liquid mixture for at least about 10 ms, or at least about 20, 30, 40, 50, 60, 70, 80, 90, 100, 150, 200, 250, 300, 350, 400, 450, 500, 600, 700, 800, 900 or 1000 ms following cessation of agitation.

A range of natural oils or synthetic essential oils, insoluble in water, can be dispersed in pure water as micron-sized droplets by the use of a freeze-thaw de-gassing process followed by vigorous shaking. These dispersions could be used for a wide range of purposes, such as for natural perfumes, skin cleaning mixtures etc. If they are stored in sealed (preferably gas-tight) vessels the dispersions may be regenerated simply by shaking, after gravity settling in the event that separation does occur. They offer a pure source of these natural oils for many applications where there is a need to remove other additives, often used to stabilize these dispersions as emulsions. Continuous agitation or freezing, after formation, will prevent gravity settling of the dispersions, so that the particle size distribution may be precisely controlled and maintained, if required. The de-gassing process may also reduce the rate at which these natural oils degrade, because of the absence of dissolved oxygen, when stored in sealed vessels.

### EXAMPLE

### Introduction

The main chemical components present in the natural oils studied in the example are given below:

Eucalyptus oil: 1,8-cineole ˜55%, α-pinene 20-30%, β-pinene ˜20-30%.
Lavender oil: 1,8-cineole 30-35%, linalool 30-40%, camphor 10-25%.
Tea tree oil: complex mixture of terpinen-4-ol, 1,8-cineole, α-pinene, α-terpinene, γ-terpinene, cis-sabinene hydrate.

The organic compounds present in each of these oil mixtures mostly cyclic, non-aromatic, hydrocarbons and linear branched chain, partially unsaturated hydrocarbons. In general they either contain no polar groups or one weakly polar group. These oil mixtures are both hydrophobic and water insoluble and so their dispersion in water might be influenced by de-gassing. In this example the effect of the de-gassing process on the dispersion of these common, natural oils was studied.

The enhancement of oil droplet dispersion in water is most easily monitored using turbidity measurements. This enhancement can be measured by the difference between the new system (degassed) and the gassed blank, following vigorous shaking. In each case, the gassed blank was a combination of the same composition as the degassed sample, but was purged with nitrogen gas via a clean pasteur pipette for ten minutes. Turbidity is a measure of how many droplets are dispersed in a given phase and is measured in NTU (nephelometric turbidity units). In the results presented here the NTU value was measured via light scattering. To give an understanding of the magnitude of these turbidity values, distilled water has a turbidity of 0.02 NTU, while tap water has a value of 1-5 NTU. Although useful, NTU measurements are of limited value and the results can be inaccurate if the refractive index of the dispersed phase is close to that of the dispersing phase. For example, visually turbid samples can give low NTU values because their opacity is caused by light absorption rather than scattering. In addition, large droplets, especially for the gassed sample, often stick to the walls of the vessel producing artificially high turbidity values. Hence, in some cases dynamic light scattering (DLS) has been used to obtain the droplet size distribution, as well as the charge on the oil droplets. Careful interpretation of the DLS results is also required for poly-disperse samples. Mono-disperse samples show size distribution by volume graphs (see later) over similar size ranges to the Z-average (diameter) and have a small PDI value (poly-dispersity index). The magnitude of the PDI is a measure of poly-dispersity and for poly-disperse samples the Z-average is accepted as the best estimate of average droplet size. PDI values of 1.0 mean that the sample is very poly dispersed and contains many large droplets.

### Materials and Methods

Water was prepared by activated charcoal and reverse osmosis filtration prior to distillation and storage in Pyrex vessels in a laminar flow filtered air cabinet. Each of the natural oils were obtained from supermarkets in the ‘purest’ form commercially available and were used without further purification. All preparations and handling of materials for these experiments were carried out in laminar flow cabinets to reduce contamination.

The surface tensions values for each of the natural oils were measured using the rod-in-free-surface (RIFS) technique (Pashley, R. M.; Karaman, M. E. Applied Colloid and Surface Chemistry, J. Wiley, New York, 2004). The RIFS technique typically gives values to an accuracy of about +/−0.1 mN/m. The interfacial tensions between natural oils and water were obtained from analysis of water droplet profiles suspended in each of the oils. This technique gave values to an accuracy of about +/−1 mN/m. The densities of these oils are all less than water. For tea tree oil, eucalyptus and lavender oil the density ranges are: 0.89-0.90, 0.91-0.93 and 0.875-0.888 g/ml, respectively.

Mixtures of various natural oils (typically about 1%) and water, and the separated liquids, were all de-gassed by a process of repeated freezing in liquid nitrogen in a tube sealed by a Teflon tap, followed by pumping down to a pressure of 0.01 mbar (with the tap open) and then melting, with the tap closed, after the tube was pumped down to the target pressure. Any dissolved gas produced on each melting cycle was removed upon re-freezing and pumping. Although this process was carried out five times, typically no further bubbling, on melting, was observed after 3-4 cycles. The vacuum pressure of 0.01 mbar corresponds to a de-gassing level of about 99.999%, if it is assumed that the final pressure achieved after several cycles of freeze/thaw/pumping is given by the pressure in equilibrium with the final frozen liquid, which on being melted does not give any visible bubbling or out-gassing.

Dispersion of oil in water was usually achieved by vigorous shaking of the mixture for 8 seconds in a sealed Pyrex tube. Turbidity was measured using an HF Scientific Micro 100 Turbidimeter. Particle sizes and zeta potentials were measured using a Malvern Zetasizer.

### Tea Tree Oil

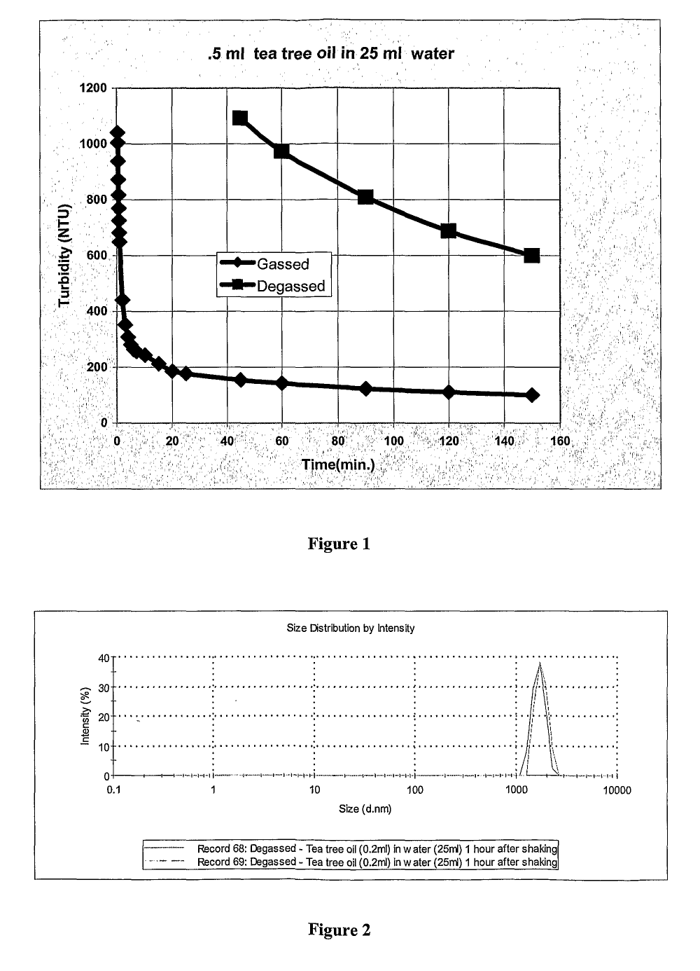

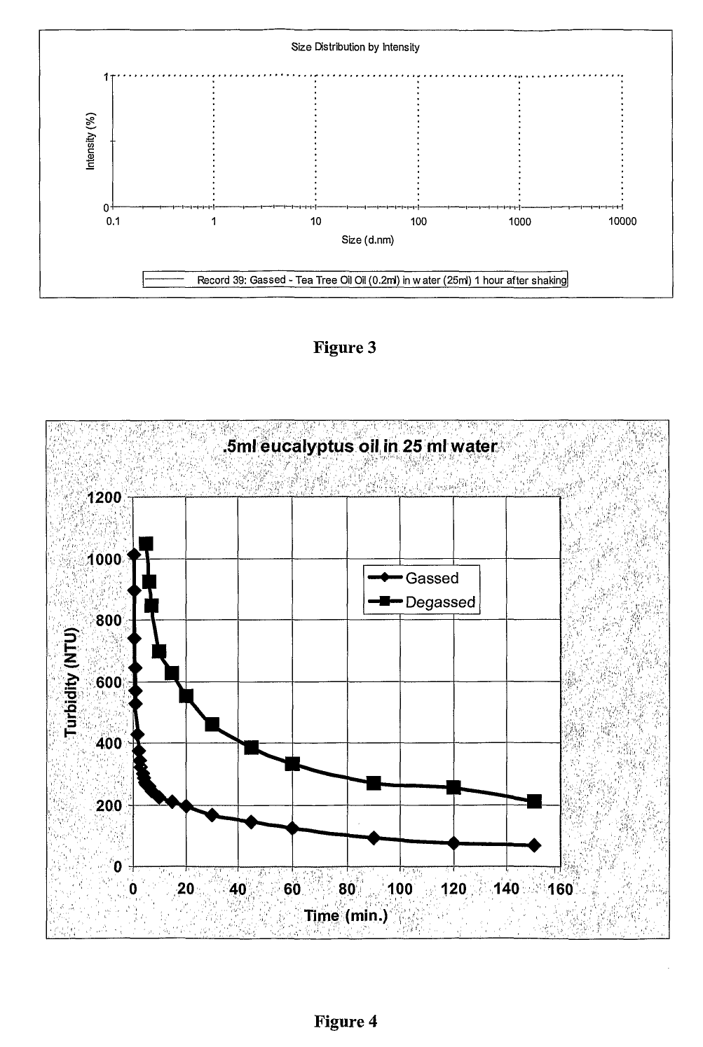

The surface tension of purchased tea tree oil was found to be 26.4 mN/m and the interfacial tension with water was 16 mN/m. Dispersions of tea tree oil and water in the range 0.2-0.5 ml in 25 ml water were studied following vigorous shaking after de-gassing or in a nitrogen gassed state (both mixtures at pH=7). The turbidity results shown in FIG. 1 clearly demonstrate the enhanced dispersion created by the de-gassing process. The blank turbidity is often artificially high because of the fact that large droplets of oil often stick to the inner walls of the glass tubes used to store the samples. Particle size measurements shown in FIGS. 2 and 3 demonstrate the fine droplets dispersed on de-gassing, with an average radius of about 0.8 μm. The dispersion is fairly mono-dispersed, whereas the gassed (blank) case cannot be measured as it contains many large droplets and is clearly poly-dispersed. Using light scattering the average zeta potential of the de-gassed dispersion droplets of tea tree oil were −35 mV.

### Eucalyptus Oil

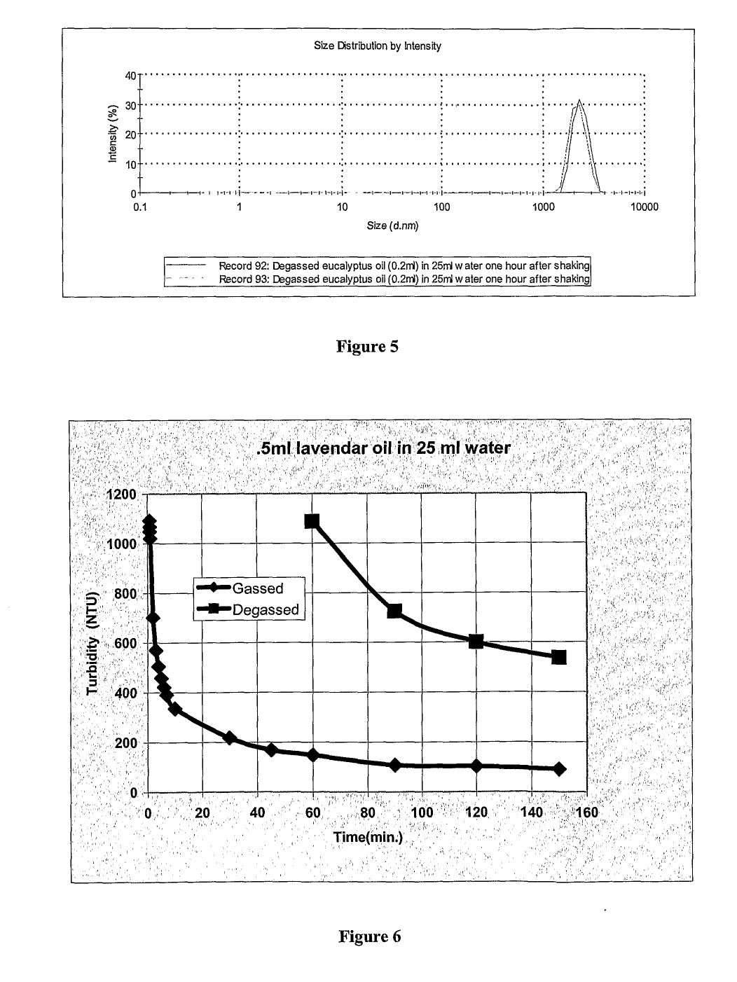

The surface tension of purchased eucalyptus oil was found to be 25.5 mN/m and the interfacial tension with water was 12 mN/m. Turbidity measurements on eucalyptus oil dispersed in water under gassed (blank) and de-gassed conditions were measured and the results shown in FIG. 4. As for the results with tea tree oil, the de-gassed mixtures gave substantially improved dispersion. Once again for the gassed case some large droplets stuck to the inside of the glass tube in which the mixtures were shaken, so the particle size distribution was also measured (FIG. 5). The de-gassed dispersion gave particles in the range of 1-2 μm radius. By comparison, the gassed samples gave polydisperse samples for which sensible values could not be obtained. The zeta potential of the de-gassed dispersion droplets was found to be −47 mV, which is close to vale reported for pure hydrocarbons.

### Lavender Oil

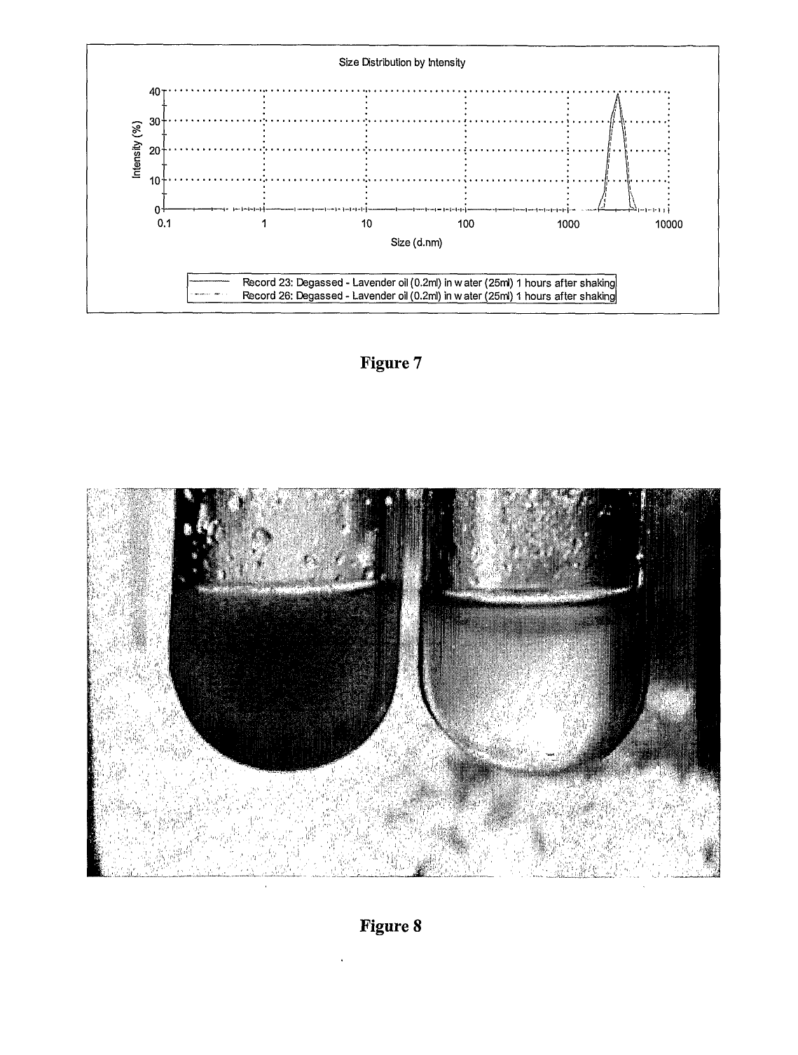

The surface tension of purchased lavender oil was found to be 25.6 mN/m and the interfacial tension with water was 13 mN/m. The effect of de-gassing on the turbidity of shaken mixtures of lavender oil and water is shown in FIG. 6. Clearly, once again de-gassing substantially increases the dispersion of this natural oil in water. Light scattering measurements of the resulting particle size distribution is shown in FIG. 7. The oil droplets are fairly mono-dispersed with an average particles radius of about 1.5 μm. The average zeta potential of these particles was found to be −41 mV, comparable with results obtained on hydrocarbon liquids. By comparison, as for the other oils studied, the gassed case gave highly poly-dispersed samples for which sensible data could not be obtained. A photograph of the effects of de-gassing on these dispersions is shown in FIG. 8.

1. A process for the production of an emulsion, comprising:
combining an aqueous liquid with a non-aqueous liquid mixture that is normally immiscible with the aqueous liquid, to form a combination;
removing dissolved gases from both the aqueous liquid and the non-aqueous liquid mixture; and
agitating the combination sufficient to form the emulsion;
wherein said combination is formed in the absence of an emulsion stabilizing agent.

2. The process of claim 1 wherein the step of combining comprises combining the aqueous liquid with the non-aqueous liquid mixture wherein said non-aqueous liquid mixture comprises at least two components having different interfacial tensions with water.

3. The process of claim 1 wherein the step of agitating comprises agitating the combination sufficient to form the emulsion such that said emulsion is stable for a period of from one hour to several weeks.

4. The process of claim 1 wherein the step of removing dissolved gases comprises removing at least 90 percent of dissolved gas present in the combination, the aqueous liquid or the non-aqueous liquid mixture.

5. The process of claim 1 wherein the step of removing dissolved gases comprises removing at least 97 percent of dissolved gas present in the combination, the aqueous liquid or the non-aqueous liquid mixture.

6. The process of claim 1 wherein removing dissolved gases comprises exposing the combination to a vacuum.

7. The process of claim 1 wherein removing dissolved gases comprises first freezing the combination and then exposing the frozen combination to a vacuum.

8. The process as in claim 6, wherein the step of exposing the combination to a vacuum comprises exposing the combination to a vacuum having an absolute pressure of less than about twenty Torr.

9. The process as in claim 6 wherein the step of exposing the combination to a vacuum comprises exposing the combination to a vacuum having an absolute pressure of less than about 0.02 Torr.

10. The process of claim 1 wherein the step of combining the aqueous liquid with the non-aqueous liquid mixture comprises combining the aqueous liquid with a non-aqueous liquid mixture which comprises at least one substance selected from the group consisting of a triglyceride and a natural oil.

11. The process of claim 1 wherein the step of combining the aqueous liquid with the non-aqueous liquid mixture comprises combining the aqueous liquid with a non-aqueous liquid mixture which does not contain soybean oil.

12. The process of claim 1 wherein the step of combining the aqueous liquid with the non-aqueous liquid mixture comprises combining the aqueous liquid with a non-aqueous liquid mixture which comprises at least one naturally occurring fragrant oil.

13. The process of claim 1 wherein the step of combining the aqueous liquid with the non-aqueous liquid mixture comprises combining the aqueous liquid with a non-aqueous liquid mixture which comprises at least one volatile component, said volatile component being selected from the group consisting of an ester, an aldehyde, an alcohol, a ketone and a terpene.

14. The process of claim 1 wherein the step of combining the aqueous liquid with the non-aqueous liquid mixture comprises combining the aqueous liquid with a non-aqueous liquid mixture which comprises an oil selected from the group consisting of eucalyptus oil, lavender oil, tea tree oil, bergamot oil, frankincense oil, patchouli oil, cedarwood oil, lime oil, peppermint oil, chamomile oil, grapefruit oil, mandarin oil, rose oil, ylang ylang oil, clary sage oil, jasmine oil, marjoram oil, cypress oil, juniper berry oil, neroli oil, rosewood oil, palmarosa oil, sandalwood oil, pine oil, mint oil, cinnamon oil, cajeput oil, fennel oil, geranium oil, girofle oil, lemon oil, spearmint oil, myrtle oil, oregano oil, rosemary oil, sarriette oil, thyme oil, hypericum oil, star anise seed oil, garlic oil and mixtures of any two or more of these.

15. The process of claim 1 wherein the step of combining the aqueous liquid with the non-aqueous liquid mixture comprises combining the aqueous liquid with a non-aqueous liquid mixture which has a lower interfacial tension with water than with air.

16. The process of claim 1 additionally comprising the step of removing water from the emulsion in order to increase the proportion of the non-aqueous liquid mixture in the emulsion.

17. An emulsion comprising a non-aqueous liquid mixture dispersed in an aqueous liquid, said emulsion having no emulsion stabilizing agent, wherein the emulsion is stable for at least one hour.

18. An emulsion produced by a process, comprising:
combining an aqueous liquid with a non-aqueous liquid mixture that is normally immiscible with the aqueous liquid, to form a combination;
removing dissolved gases from both the aqueous liquid and the non-aqueous liquid mixture; and
agitating the combination sufficient to form the emulsion;
wherein said combination is formed in the absence of an emulsion stabilizing agent.

19. An emulsion according to claim 17, said emulsion having a mean droplet size of less than about 10 microns.

20. The emulsion of claim 17 wherein the proportion of the non-aqueous liquid mixture in the emulsion is between about 0.1 and about 50% w/w or w/v.

21. A preparation for use in maintaining or improving personal hygiene or for use as a cosmetic, said preparation comprising an emulsion according to claim 17.

22. An apparatus for preparing an emulsion of a non-aqueous liquid mixture in an aqueous liquid, said apparatus comprising:
a mixing chamber for combining the aqueous liquid with the non-aqueous liquid mixture to form a combination;
at least one degasser for removing dissolved gas from both the aqueous liquid and the non-aqueous liquid mixture; and
an agitator for agitating the combination to form the emulsion.
